(19) United States
(12) Patent Application Publication
(10) Pub. No.: US 2015/0056314 A1
(43) Pub. Date: Feb. 26, 2015

(54) EXTRACTION METHOD FOR USE IN EXTRACTING BENEFICIAL COMPOUNDS FROM COFFEE BEANS

(71) Applicant: Applied Food Sciences, Inc., Austin, TX (US)

(72) Inventor: Christine C. Fields, Lake Zurich, IL (US)

(21) Appl. No.: 14/530,083

(22) Filed: Oct. 31, 2014

Related U.S. Application Data
(62) Division of application No. 12/913,745, filed on Oct. 27, 2010.
Application No. 12/913,745 is a division of application No. 10/458,754, filed on Jun. 11, 2003.

(51) Int. Cl. A61K 36/74; A61K 31/192; A61K 31/216

(52) U.S. Cl. CPC A61K 36/74; A61K 31/216; A61K 31/192
USPC 424/776; 514/533

(57) ABSTRACT

An improved method for producing a coffee bean extract containing beneficial compounds.

### CROSS-REFERENCE TO RELATED APPLICATIONS

This application is a divisional of U.S. application Ser. No. 12/913,745, filed Oct. 27, 2010, which is a divisional of U.S. application Ser. No. 10/458,754, filed Jun. 11, 2003, which is herein incorporated by reference their entirety.

### BACKGROUND OF THE INVENTION

1. Field of the Invention

This invention relates to a new method for optimizing beneficial extracts from coffee beans.

2. Background Information

Phenolic compounds are known antioxidants and anti-tumor agents, and are present in coffee beans, mainly esters of quinic acid with different amount of caffeyl groups attached to its different positions. The phenolic acids present in coffee such as chlorogenic acid, caffeic acid, para-coumaric acid and eugenol have been shown to exert cancer preventive activities in animal models. Chlorogenic acid has also been found to inhibit methylazoxymethanol-induced large intestinal tumors in hamster models.

Chlorogenic acid, an ester of caffeic acid and quinic acid, is the main phenolic acid in coffee. Chlorogenic acid is known to protect the gastric mucosa against irritations, and, therefore, improves the digestibility of foods, beverages and medicaments. The improved digestibility is expressed through a much-reduced systemic acid secretion (such as causes heartburn, etc.), which has been found to be directly dependent on an increased level of chlorogenic acid content in raw green coffee beans.

Chlorogenic acid also is an antioxidant in vitro and will be useful in vivo in humans to contribute to the prevention of cardiovascular disease. In vivo assays show that one third of chlorogenic acid and almost all caffeic acid is absorbed in the small intestine of humans. This implies that part of the chlorogenic acid from foods will enter into the blood circulation, but most will reach the colon. Caffeic seems to be more bioavailable. (Olthof et al J Nutr 2001 January; 131 (1):66-71).

Further still, chlorogenic acid has a chemopreventative effect on rat stomach cancer. (J Toxicol Sci 1999 December; 24 (5):433-9 Shimizu et al).

Finally, extract from coffee beans can also be used as a preservative or as a topical skin treatment in cosmetic or pharmaceutical preparations.

Clearly, it is desirable to make the beneficial compounds of coffee conveniently available for human consumption. Such availability requires, as a practical matter, that it not be limited to the consumption of coffee in beverage form. Coffee beverage consumption as a sole means of accessing coffee's beneficial compounds would, in many cases, require the consumption of more than a desirable amount of coffee in order to reach optimum intake levels, even for those who enjoy coffee. However, many people simply do not like coffee, and, by avoiding its consumption, would forego its highly beneficial phenolic constituents.

An extract from coffee beans (a powder when produced according to the present invention shown below) is the answer. No beverage consumption is required, as the extract can be encapsulated, tableted, or placed in any other type of acceptable pharmaceutical carrier for consumption by humans or animals as a dietary supplement, as a constituent of preservatives, cosmetic creams, or in any number of other contexts in which the beneficial chemical behavior of the extract constituents are desirable.

As in the production of any consumable, efficiency of production is of paramount importance, both to the producer and the consumer. Less efficiency means higher production costs and higher consumer prices. In the case of health-promoting products, the disincentive to purchase products which accompanies higher prices is doubly regrettable because of the suffering that a foregone purchase might have prevented.

It is highly desirable, therefore, to provide a method which extracts the beneficial compounds in coffee, not only to the greatest degree possible in the absolute quantity sense, but does so a more cost effective manner than any presently known coffee extraction process.

### SUMMARY OF THE INVENTION

It is an object of the present invention to provide an improved process for extracting beneficial compounds from coffee beans.

It is another object of the present invention to provide a method which extracts a higher percentage of beneficial compounds from coffee beans than presently known methods.

It is another object of the present invention to provide a method for extracting beneficial compounds from coffee beans in a more cost effective manner than presently known coffee bean extraction methods.

It is another object of the present invention to provide a method which both extracts a higher percentage of beneficial compounds from coffee beans than presently known methods, and does so in a more cost effective manner than such presently known coffee bean extraction methods.

It is another object of the present invention to provide a product by process, which product is a coffee bean extract which can be taken in a pharmaceutical carrier context, or as a dietary supplement in foods and beverages, in either event providing beneficial compounds derived from coffee beans and providing anti-oxidant and other health benefits, including, but not limited to, therapeutic and prophylactic treatments for hypertension, diabetes, weight management, depression and sexual dysfunction.

In satisfaction of these and related objects, the present method for extracting beneficial compounds from coffee beans which produces a higher yield of such compounds, and a production cost which is substantially more desirable than presently known or employed coffee bean extraction methods. The present invention also encompasses products yielded through the present process.

The present method employs a particular series of steps and reagents which, in combination, yields the aforesaid benefits.

Upon adoption of the present method, industry will be able to provide to the public a health-promoting compound in a format which is affordable, does not require the consumption of a particular food or beverage to realize its benefits. The end product—an innocuous powder, is easily incorporated into a wide array of products to a highly beneficial result, yet, in some contexts, such as in the case of use as a food additive, an effectively unnoticeable form.

### DETAILED DESCRIPTION OF THE PREFERRED EMBODIMENT

The following methodology has been determined by the present inventors to both: (1) obtain a higher yield of beneficial compounds from coffee beans than any industry method for coffee bean extraction; and (2) obtain such yield at a cost efficiency greater than the presently lower yielding methods.

### Substrate Preparation

Optimal extraction yield is obtained first by grinding any species of sun-dried coffee beans. It is preferable to use coffee beans which are dried other than through conventional roasting (sun-dried is the preferred mode of the present invention) as the roasting process substantially diminishes the chlorogenic acid content of coffee beans.

Substantial experimentation has revealed that coffee bean particle size is important to the efficiency (performance and cost) in the extraction process. Particles processed for passing a 20 mesh sieve have been determined to be optimal. Certain temperature parameters indicated below have also been shown to enhance the yield of the over-all extraction process.

The recommended equipment for producing the ground substrate needed for the present extraction process is as follows:

Hammer mill, output 100 kgs/hr.

Sifter equipped with a 5 and a 20 mesh sieve

Thermometer calibrated to 1000° C.

The grinding procedure is as follows: Load the coffee beans into the hammer mill and actuate the mill. Ensure that the temperature of the hammer mill (or “pulverizer”) does not exceed 80° C. If the temperature exceeds 80° C., stop the operation till the temperature drops down to 40° C. Sift the pulverized powder through 5 mesh screen and collect the powder into a plastic container. Grindings retained on the 5 mesh screen should be kept separately. The grindings that have passed through the 5 mesh screen are sent to a second pulverization stage, and processed for passing through a 20 mesh sieve.

The retentate on the 20 mesh sieve should be collected separately. The retentate on 5 mesh sieve should be pulverized further to give the desired particle size of 20 mesh. The retentate on 20 mesh should also be pulverized separately to get a particle size to pass through 20 mesh.

### Extraction

Once the substrate for the extraction process has been produced, as indicated above, the actual extraction process proceeds with the following equipment requirements (scaled for the indicated amount of substrate, with scaling changes dictated by industry application need as will be apparent to persons skilled in the relevant arts):

800-liter extractor equipped with condenser, anchor type stirrer, condenser, receiver, and a reactor false bottom with a 40 mesh plate above the drain valve.

Nutsch filter

The required reagents (again, scalable to industry needs) include 100 kgs of coffee bean particles; 1200 liters of methanol, 2000 liters of DM water, and 400 liters of N Hexane.

The preferred extraction procedure beings by cleaning the reactor thoroughly with DM water and draining the washings completely. The reactor jacket is then heated to 80° C. to drive out any remaining water, after which the jacket is returned to room temperature.

Coffee bean powder and N Hexane are placed in the reactor, stirred for two hours at room temperature, and filtered with the nutsch filter. The residue is re-inserted into the reactor and the N Hexane wash and filtration steps are repeated.

The resulting residue is then air dried to a Loss on drying level of 10%

The residue of the preceding step is next placed back into the reactor for extraction, along with 240 liters of DM water and 360 liters of methanol. The reactor jacket is heated to 60°-65° C. and the mass is maintained at 50°-55° C. over a four hour extraction phase. After the four hour extraction, heat is removed from the reactor jacket, and it is allowed to cool to room temperature.

The mass from the reactor is then filtered with the Nutsch filter, placed back into the reactor, and the four hour extraction, cooling and filtration is repeated a second time.

### Concentration

The extract produced to this point in the process is then placed in a second reactor in a concentration step.

The concentration step involves the following equipment (proportionately scaled as appropriate):

3 KL reactor, equipped with anchor type of stirrer, condenser and receiver.

Watering vacuum pump

Water ejector

The concentration step then proceeds as follows:

Placing the extract into the reactor and closing the lid, one sets the jacket temperature to about 50° C. A vacuum of 650 mm Hg is applied, and the solvent stripping commences, ensuring that the vacuum is at all times maintained at 650 mm Hg, and the mass temperature does not exceed 55° C. One next concentrates the water, after removal of the solvent, to volume of 60 liters.

The mass is then drained from the reactor and relocated for drying.

### Drying

The final, drying step involves a spray drier with a water evaporation rate of 3-5 kgs per hour. The dryer should be equipped with a variable RPM rotating disc, an inlet for nitrogen purging, an air compressor having a- capacity of 10 kgs. Stainless steel collection bins, each having a capacity of 5 kgs, are required for collection of the final product.

The drying step proceeds as follows: The spray drier is to be cleaned with DM water and dried using hot air. The inlet temperature is to be set at 180° C., and the outlet temperature should be set at 120° C.

The dryer disc RPM is set to 4000, the system is purged with dry nitrogen, the air pressure to 5 kgs, and the concentrated extract is processed to a final, fine powder end-product.

### Best Mode and Variations

The process, substrate and reagents shown above are considered optimal. Certain variations in the above will clearly reside within the scope of the present invention. However, the rationale behind certain choices of reagents, material characteristics, etc. warrant comment.

Optimal extraction was observed when the coffee bean particle size was 20 mesh or below, but the particle size was lowered than 30 mesh, the filtration time post extraction took a considerably longer time. Therefore, the particle size was optimized to 20 mesh.

The bean pulverization is through a hammer mill, rather than ribbon blade unit, because of the hard nature of the bean, due to the tough polysaccharide layer in the epidermis.

The extraction process is designed for optimal extraction of total chlorogenic acids, with a special emphasis on the extraction of the 5-HCA isomer. Also, the oils and flavor agents in the coffee bean are to be removed, to produce the least user perceptible end-product possible.

The oils and terpenes can be removed using either N Hexane or petroleum ether, but N Hexane proved to be a better agent in view of its effect on the terpenes.

Extraction of the residue, obtained after the removal of oil and terpenes may be extracted using a combination of polar to intermediately polar solvents in combination with water, with the objective of minimal polysaccharides extraction. The use of methanol and water in a volume ratio of 60:40 provided the best extractive value for total chlorogenic acid. Use of solvents like methanol ensured that the polysaccharides were not extracted in larger proportion. However, using pure methanol did not improve the purity of chlorogenic acids, because methanol also showed solvation for some of the intermediately polar compounds.

The use of a combination of acetone and water, or isopropyl alcohol and water, did not show the same extraction efficiency as methanol and water system in terms of yield and chlorogenic acid content.

The extraction occurs at a temperature of 50°-55° C., because, at higher temperatures, there is a distinct drop in the chlorogenic acid content.

The down steam processing of the methanol and water extraction involves filtration. Fastest filtration occurs through pressure filtration using a plate frame type filter, but Nutsch filtration appears to be the most practical selection. Sparkler filtration slows the over-all process, with clogged sparkler leaves from the mucilaginous impurities present in the extract.

### Analysis

Analysis of the extract was conducted to prove the concept.

The coffee bean extract was subjected to analysis using HPLC. The extract was dissolved in HPLC grade water and was injected on to a C-18 column (25 cms×4.5 mm id, particle size 5 microns, pore size 100 Ao). The mobile phase used was a gradient of acetonitrile (containing 1% acetic acid) and water (containing 1% acetic acid). The analysis was done with respect to assay of chlorogenic acid against a reference standard of Chlorogenic acid. The percentage of chlorogenic acid was expressed as absolute assay as well as in terms of its chromatographic purity (based on area normalization method). The detection was done using a UV detector set at 320 nm.

Column: 250 mm×4.6 mm id, hypersil C 18, particle size 5 microns, pore size 100 Å.

Mobile: A: 1% acetic acid in acetonitrile

Phase: B: 1% acetic acid in water

Gradient from 10% of A to 30% of A over 10 minutes and back to 10% of A after 15 minutes.

Flow Rate: 0.7 ml/min.

Detector: U.V. set at 320 nm

Standard Concentration: Chlorogenic acid, 400 μg/ml, prepared in water

Sample Concentration: 500 μg/ml prepared in water

Sample Size: 20 μl

A) Preparation of Mobile Phase
Solvents: Water, HPLC grade
   Acetonitrile, HPLC grade,
   Acetic acid, AR grade
Apparatus: 500 ml reagent bottle, ultrasonic bath, membrane filtration system, 500 ml measuring cylinder, 5 ml bulk pipette, 1 liter beaker.
Solvent A: Measure accurately 500 ml of HPLC grade acetonitrile, transfer it into a 1 liter beaker. Add to it 5 ml of glacial acetic acid. Stir well and filter through 0.22μ membrane filter. Sonicate it on an ultrasonic bath for 15 minutes.
Solvent B: Measure accurately 500 ml of HPLC grade water and transfer into a 500 ml beaker. Add to it 5 ml of glacial acetic acid. Stir well and filter through 0.22μ membrane filtration system. Sonicate on an ultrasonic bath for 15 minutes.

B) Preparation of Solutions:
Apparatus: 5 ml standard volumetric flask, 50 ml standard volumetric flask, syringe based membrane filtration system.
Solvents: HPLC grade water
Preparation of Standard Solution:
   Weigh accurately 2 mg of chlorogenic acid standard and transfer it into a standard volumetric flask. Dissolve in 2 ml of HPLC grade water. Dilute up to the mark using HPLC grade water. The standard solution has a strength of 400 μg/ml. Filter through a 0.22μ membrane filter using a syringe based system.
Preparation of Sample Solution:
   Weigh accurately 25 mg of the coffee bean extract and transfer it into a 50 ml standard volumetric flask. Dissolve the product in 30 ml of HPLC grade water and dilute up to to the mark using HPLC grade water. The sample solution has a concentration of 500 μg/ml.
   Set the operating conditions. Set the detector at 320 nm. Set the gradient table on the instrument as given below:

| | A | B |
|---|---|---|
| Gradient | 10 | 90 |
| Time | % A | Flow |
| 0.00 | 10.0 | 0.7 |
| 5.00 | 30.0 | 0.7 |
| 10.00 | 30.0 | 0.7 |
| 15.0 | 10.0 | 0.7 |
| 20.00 | 10.0 | 0.7 |
| 25.00 | 10.0 | 0.7 |

Flush the column with HPLC grade water at a flow rate of 1 ml per minute for a period of 30 minutes. Flush the column with HPLC grade Methanol for 30 minutes. Run the gradient as per the conditions as given in the above table. Run a blank gradient. Note the signals for a blank run.
Inject the standard solution into the system. Note the retention time and peak area. Inject the standard 5 times and perform the system suitability test (the relative standard deviation for area counts should not be more than 1.5%) Determine the average area for 5 injections.
Inject the sample solution into the system. Note the retention time for 5-HCA from the sample. Repeat the injection 5 times and perform the system suitability test for the sample. Calculate the mean area for 5-HCA from the sample.

C) Calculation
Weight of the Standard Taken: Wstd
Weight of the Sample Taken: Wsa
Area Corresponding to: Astd
5-HCA from standard Area Corresponding: Asa
To 5-HCA from Sample

Dilution of Standard: Dstd

Dilution of Sample: Dsa

Purity of standard: P

$$Assay purity of 5 H C A from sample = \frac{Weight of of std .}{Weight of sa .} \times \frac{Area of Sa}{Area of Std .} \times \frac{Dilution Purity of Sa}{Dilution of Std .} \times PURITY \times 100$$

$$\frac{Wstd}{Wsa} \times \frac{Asa}{Astd} \times \frac{Dsa}{Dstd} \times P \times 100$$

Chromatographic purity of 5-HCA is calculated as:
Perform baseline correction with respect to blank run. Eliminate the peaks corresponding to solvents Calculate the total peak area for all the signals recorded on the chromatogram excluding the signals for the peaks corresponding to the solvent. This total peak area is taken as Pt. Identify the peak for 5HCA from the sample and it is taken as PHCA.

Percentage chromatographic purity is given as:

$$\frac{P H C A}{Pt} \times 100$$

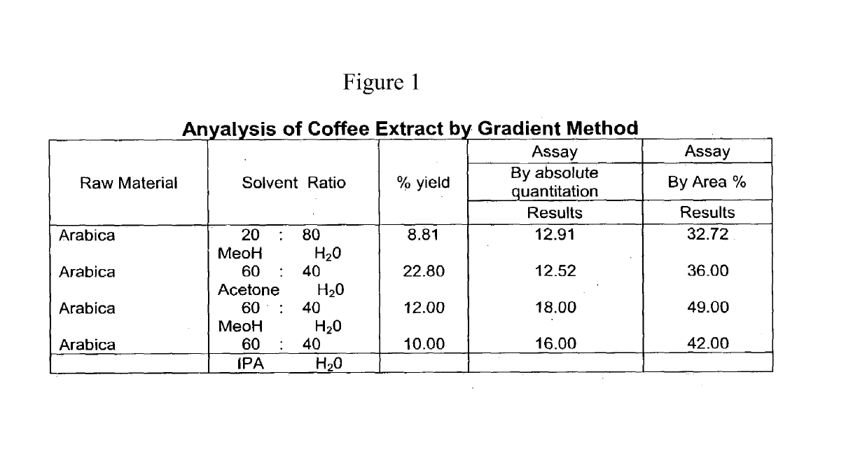

Analysis results are shown in FIG. 1 and reveal an improved yield of beneficial chlorogenic compounds, when compared to present coffee bean extraction methods.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

What is claimed is:

1. A method of producing an extract of coffee bean substrate, the method comprising the steps of:
grinding coffee beans to produce a particulate;
substantially removing oils and terpenes from said particulate with an intermediate solvent;
extracting beneficial chlorogenic compounds from said particulate through application of polar or intermediately polar solvents; and
stripping solvents from the product of said extracting.

2. The method of claim 1, wherein a hammer mill is used for the step of grinding the coffee beans.

3. The method of claim 1, wherein the step of extraction occurs at a temperature of 50-55° C.

4. The method of claim 1, wherein the intermediary solvent is chosen from N Hexane or petroleum ether.

5. The method of claim 1, wherein the polar or intermediary polar solvents comprises methanol and water in a volume ratio of 60:40.

6. The method of claim 1, further comprising substantially removing oils and terpenes from said particulate.

7. The method of claim 2, wherein said particulate is ground to produce particles of approximately a 20 mesh sieve passage size.

8. The method of claim 7, wherein the step of extraction occurs at a temperature of 50-55° C.

9. The method of claim 8, wherein the polar or intermediary polar solvents comprises methanol and water in a volume ratio of 60:40.

10. The method of claim 9, wherein the intermediary solvent is chosen from any of N Hexane, petroleum ether, and combinations thereof.

11. The method of claim 10, further comprising the step of substantially removing oils and terpenes from said particulate.

12. The method of claim 11, wherein said product is the extract of coffee comprising 15%-26% 5-CQA, 5-O-caffeoylquinic acid.

13. The method of claim 11, wherein the step of extraction occurs in a reactor and a reactor jacket is heated to a temperature of 60-65° C.

14. The method of claim 13, wherein said product is the extract of coffee comprising 15%-26% 5-CQA, 5-O-caffeoylquinic acid.

15. A method of producing an extract of coffee bean substrate, the method comprising the steps of:
grinding coffee beans to produce a particulate;
substantially removing oils and terpenes from said particulate with an intermediate solvent, wherein the intermediary solvent is chosen from N Hexane or petroleum ether;
extracting beneficial chlorogenic compounds from said particulate through application of polar or intermediately polar solvents;
stripping solvents from the product of said extracting;
concentrating the product; and
drying the product.

16. The method of claim 15, wherein the step of drying comprises of applying a vacuum of 650 mm Hg.

17. The method of claim 15, wherein the polar or intermediary polar solvents comprises methanol and water in a volume ratio of 60:40.

18. The method of claim 17, wherein said product is the extract of coffee comprising 15%-26% 5-CQA, 5-O-caffeoylquinic acid.

19. A method of producing an extract of coffee bean substrate, the method comprising the steps of:
grinding coffee beans to produce a particulate;
substantially removing oils and terpenes from said particulate with an intermediate solvent, wherein the intermediary solvent is chosen from N Hexane or petroleum ether;
extracting beneficial chlorogenic compounds from said particulate through application of polar or intermediately polar solvents, wherein the polar or intermediary polar solvents comprises methanol and water in a volume ratio of 60:40; and
stripping solvents from the product of said extracting.

20. The method of claim 19, wherein the step of extraction occurs at a temperature of 50-55° C. over a phase of four hours.

21. The method of claim 20, wherein said product is the extract of coffee comprising:
from 15%-26% of 5-CQA, 5-O-caffeoylquinic acid;
from 1%-6% of 3-CQA, 3-O-caffeoylquinic acid;
from 4%-16% of 4-CQA, 4-O-caffeoylquinic acid;
from 2%-12% of 3,4-dicaffeoyl quinic acid;
from 1%-12% of 3,5-dicaffeoyl quinic acid;
from 2%-14% of 4,5-dicaffeoyl quinic acid;
from 1%-10% of ferulic acid; and
from 1%-10% of p-coumaric acid.
